(19) United States
(12) Patent Application Publication
(10) Pub. No.: US 2013/0237995 A1
(43) Pub. Date: Sep. 12, 2013

(54) SURGERY ROBOT SYSTEM, SURGERY APPARATUS AND METHOD FOR PROVIDING TACTILE FEEDBACK

(71) Applicant: SAMSUNG ELECTRONICS CO., LTD., Suwon-si (KR)

(72) Inventors: Hyung Kew LEE, Gunpo-si (KR); Joon-Ah PARK, Seoul (KR)

(73) Assignee: SAMSUNG ELECTRONICS CO., LTD., Suwon-si (KR)

(21) Appl. No.: 13/848,315

(22) Filed: Mar. 21, 2013

Related U.S. Application Data
(62) Division of application No. 13/137,777, filed on Sep. 12, 2011.

(30) Foreign Application Priority Data
Sep. 17, 2010 (KR) 10-2010-0091508

(51) Int. Cl. A61B 19/00

(52) U.S. Cl. CPC A61B 19/2203
USPC 606/130

(57) ABSTRACT

A surgery robot system provides tactile feedback. The surgery robot system includes a master robot and a slave robot mounted with a surgery tool including at least one tactile sensor that generates a tactile signal upon contact with a surgery region. The master robot is adapted to generate a control signal to control operation of the surgery tool and to receive and reproduce the tactile signal from the slave robot.

### CROSS-REFERENCE TO RELATED APPLICATIONS

This application is a divisional of U.S. patent application Ser. No. 13/137,777, filed on Sep. 12, 2011 and which is pending, and claims the benefit of Korean Application No. 2010-0091508, filed on Sep. 17, 2010, in the Korean Intellectual Property Office, the disclosures of which are incorporated herein by reference.

### BACKGROUND

1. Field

Example embodiments of the following description relate to a surgery robot system, a surgery apparatus, and a method for providing a tactile feedback.

2. Description of the Related Art

In medical terminology, surgery is an operation to treat a disease or disorder, for example, by cutting or incising skin, mucosa, or other tissues by using a medical machine. In particular, open abdominal surgery performed by cutting open abdominal skin and treating, forming, or removing internal organs may cause various problems such as bleeding, side effects, patient pain, and scars. Accordingly, use of surgery robots has recently increased to minimize bleeding and patient pain.

When a surgery robot is used, a surgeon may be able to directly check small vessels and nerves, and avoid even a minor hand tremor. That is, a precise and stable surgery may be performed. Such characteristics of the surgery robot have enabled successful surgeries for prostate cancer, bladder cancer, renal pelvis cancer, colon cancer, and the like.

The surgery robot is operated by a master-slave system. In further detail, as a surgeon operates a master robot, the master robot generates and transmits a control signal to a slave robot. Accordingly, the slave robot operates and performs surgery on a patient based on the control signal. The surgeon is able to monitor a state of the surgery through the master robot. However, the surgeon is not in direct contact with the patient and, therefore, cannot perform palpation during the surgery. That is, since the surgeon is unable to perceive a degree of contact between a surgery tool mounted to the slave robot and a surgery region of the patient, tissues of the surgery region may be pinched or pulled and thus may be damaged. In addition, the surgeon is unable to detect abnormal tissues through palpation.

### SUMMARY

According to example embodiments, there may be provided a surgery robot system enabling generation of a tactile signal by detecting contact between a surgery tool and a surgery region and reproducing the tactile signal, thereby providing a tactile feedback, and also provided are a surgery apparatus and a method of providing the tactile feedback.

The foregoing and/or other aspects are achieved by providing a surgery robot system including a slave robot mounted with a surgery tool including at least one sensor that generates a contact signal upon contact with a surgery region; and a master robot adapted to generate a control signal to control operation of the surgery tool and to receive and reproduce the contact signal from the slave robot.

The slave robot may include a signal converter to convert the contact signal generated from the sensor into an electrical signal; a slave transceiver to receive the control signal from the master robot and to transmit the electrical signal converted from the contact signal to the master robot; and a slave controller to control operation of the surgery tool in accordance with the received control signal.

The slave robot may further include a temperature sensor to detect a variation of temperature of the surgery tool and to generate a temperature signal; and a signal combiner to combine the temperature signal with the electrical signal converted from the contact signal.

The sensor may be a tactile sensor which may detect a degree of contact between the surgery tool and the surgery region according to a mechanical deformation of the surgery tool, and may generate the contact signal according to the contact degree being detected, the contact signal being a tactile signal.

The surgery tool may include a plurality of tactile sensors as the at least one sensor and may include a cylindrical portion extending from one end thereof, and when the plurality of tactile sensors are provided to the surgery tool, the tactile sensors may be symmetrically arranged on an inner surface or an outer surface of the cylindrical portion.

The master robot may include a user operator to generate a control signal to control operation of the surgery tool in accordance with a user operation; a master transceiver to transmit the control signal to the slave robot and to receive the contact signal from the slave robot; a signal adjuster to adjust a signal level of the received contact signal, so that the contact signal is processable by the master robot; a tactile reproduction actuator in the user operator; and a master controller to drive the tactile reproduction actuator to reproduce the signal level-adjusted contact signal.

The master robot may include a temperature value generator in the user operator, the temperature value generator to separate the temperature signal from the contact signal received by the master transceiver, and to generate a temperature value corresponding to the temperature signal; and a display to display the signal level-adjusted contact signal and the separated temperature signal.

The tactile reproduction actuator may include any one selected from a pneumatic actuator, a piezoelectric actuator, and a shape memory alloy (SMA) actuator.

The foregoing and/or other aspects are achieved by providing a surgery apparatus including a surgery tool; a user operator to generate a control signal to control operation of the surgery tool in accordance with a user operation; at least one sensor to generate a tactile signal upon contact between the surgery tool and a surgery region; and a tactile reproduction actuator to reproduce the tactile signal.

The at least one sensor may be a tactile sensor which may be a fiber optic sensor included in the surgery tool to detect a degree of contact between the surgery tool and the surgery region according to a mechanical deformation of the surgery tool, and to generate the tactile signal according to the degree of contact being detected.

The surgery apparatus may further include a signal converter to convert the tactile signal generated by the at least one sensor into an electrical signal.

The surgery apparatus may further include a temperature sensor to detect a variation of temperature of the surgery tool and to generate a temperature signal; a temperature value generator included in the user operator to generate a temperature value corresponding to the generated temperature signal; and a display to display the tactile signal and the temperature signal.

The foregoing and/or other aspects are achieved by providing a method of providing tactile feedback including generating a control signal to control operation of a surgery tool; operating the surgery tool in accordance with the control signal; generating a contact signal using at least one sensor included in the surgery tool when the surgery tool contacts a surgery region; and reproducing the contact signal.

The generating the tactile signal may include converting the contact signal generated from the at least one sensor into an electrical signal.

The generating the contact signal may include detecting a variation of temperature of the surgery tool by a temperature sensor and generating a temperature signal.

The generating the contact signal may include detecting a degree of contact between the surgery tool and the surgery region according to a mechanical deformation of the surgery tool, and generating a tactile signal as the contact signal according to the degree of contact being detected.

The reproducing the contact signal may include generating a temperature value corresponding to the temperature signal by driving a temperature value generator; and displaying the contact signal and the temperature signal.

Additional aspects, features, and/or advantages of example embodiments will be set forth in part in the description which follows and, in part, will be apparent from the description, or may be learned by practice of the disclosure.

### BRIEF DESCRIPTION OF THE DRAWINGS

These and/or other aspects and advantages will become apparent and more readily appreciated from the following description of the example embodiments, taken in conjunction with the accompanying drawings of which:

FIG. 1 is a diagram of a surgery robot system providing tactile feedback, according to example embodiments;

FIG. 2 is a block diagram of a structure of the surgery robot system of FIG. 1;

FIG. 3 is a block diagram of a structure of a surgery apparatus according to example embodiments;

FIG. 4 is a diagram of a surgery tool that generates a tactile signal of the surgery robot system of FIG. 1;

FIGS. 5, 6 and 7 are diagrams of various types of the tactile sensors for the surgery tool;

FIG. 8 is a diagram of a user operator that provides tactile feedback, according to example embodiments;

FIG. 9 is a flowchart illustrating a method for providing tactile feedback according to example embodiments; and

FIG. 10 is a flowchart illustrating a method for providing tactile feedback according to other example embodiments.

### DETAILED DESCRIPTION

Reference will now be made in detail to exemplary embodiments examples of which are illustrated in the accompanying drawings. In the following description if detailed descriptions of related disclosed art or configurations have been determined to unnecessarily make the subject matter of the embodiments obscure, they are omitted. Terms to be used below are defined based on their functions in the present embodiments and may vary according to users, user's intentions, or practices. Therefore, the definitions of the terms should be determined based on the entire specification.

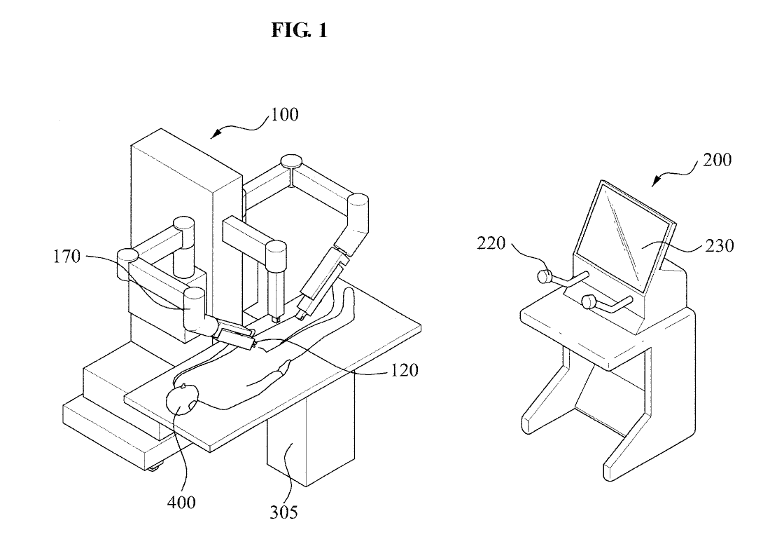

FIG. 1 is a diagram showing a surgery robot system providing tactile feedback, according to example embodiments. Referring to FIG. 1, the surgery robot system employs a master-slave system which includes a slave robot 100 and a master robot 200.

When a surgeon operates a user operator or user actuation device 220 mounted on the master robot 200, the master robot 200 generates a control signal and transmits the control signal to the slave robot 100. Receiving the control signal, the slave robot 100 controls operation of a surgery tool 120.

The slave robot 100 includes the surgery tool 120. The surgery tool 120 is connected to a main body of the slave robot 100 by a robot arm 170. The slave robot 100 controls the operation of the surgery tool 120 according to the control signal received from the master robot 200, and therefore performs surgery with respect to a patient 400 on an operating table 305. That is, the surgery tool 120 performs the surgery through incision, suture, and the like, in direct contact with the surgery region of the patient 400.

During the surgery, the slave robot 100 generates a contact signal, such as a tactile signal, using at least one sensor, such as a tactile sensor, provided on the surgery tool 120. When a mechanical deformation of the surgery tool 120 occurs by contact between the surgery tool 120 and the surgery region, the tactile sensor detects the deformation and accordingly generates the tactile signal.

The slave robot 100 adjusts a signal level of the tactile signal so that the tactile signal is processable by the master robot 200, and transmits the level-adjusted tactile signal to the master robot 200. Upon receiving the tactile signal from the slave robot 100, the master robot 200 reproduces the tactile signal using a tactile reproduction actuator provided to the user operator 220. Accordingly, the surgeon may be provided with tactile feedback corresponding to the contact between the surgery tool 120 and the surgery region while, at the same time, performing the surgery by operating the user operator 220. Additionally, the master robot 200 may display the tactile signal through a display 230 in the form of text or a graph.

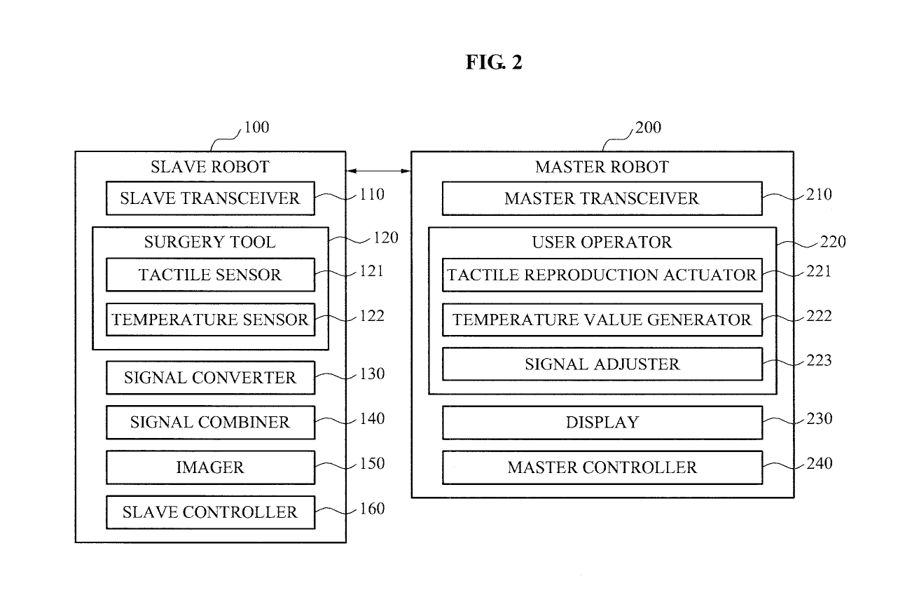

FIG. 2 is a block diagram illustrating a structure of the surgery robot system of FIG. 1. Referring to FIG. 2, the surgery robot system includes the slave robot 100 and the master robot 200. The slave robot 100 includes a slave transceiver 110, the surgery tool 120, a signal converter 130, a signal combiner 140, an imager 150, and a slave controller 160.

The slave transceiver 110 transmits and receives signals to and from the master robot 200. The slave transceiver 110 receives a control signal for controlling operation of the surgery tool 120, from the master robot 200. The surgery tool 120 operates in accordance with the control signal received from the master robot 200, thereby performing surgery on a patient. The surgery tool 120 includes a tactile sensor 121 and a temperature sensor 122.

While the surgery tool 120 is performing the surgery, the tactile sensor 121 detects contact between the surgery tool 120 and the surgery region of the patient 400 and accordingly generates the tactile signal. Specifically, the tactile sensor 121 detects a degree of contact between the surgery tool 120 and the surgery region according to a mechanical deformation of the surgery tool 120 caused by the contact with the surgery region, and generates the tactile signal corresponding to the degree of contact being detected. Therefore, the tactile sensor 121 detects not only contact such as pressing and touching the surgery region by the surgery tool 120 but also grabbing or pulling of the surgery region by the surgery tool 120, and correspondingly generates the tactile signal.

The tactile sensor 121 may be a fiber Bragg grating (FBG) sensor which is a type of fiber optic sensor. The FBG sensor is structured by engraving a plurality of fiber optic Bragg gratings on one strand of fiber optic in uniform lengths. The FBG sensor detects strength or temperature based on variation of a wavelength of light reflected from the respective gratings according to environmental factors such as the strength or temperature. When a mechanical deformation occurs, the plurality of fiber optic Bragg gratings constituting the FBG sensor are changed in refractive index or length, thereby changing the wavelength of light reflected from the respective gratings. Therefore, the FBG sensor measures the wavelength of light reflected from the plurality of fiber optic Bragg gratings while the surgery tool 120 is performing the surgery. When the wavelength of light from some of the Bragg gratings is different from a reference wavelength, the FBG sensor may correspondingly generate the tactile signal.

The FBG sensor may be disposed in close contact with an inner surface or an outer surface of the surgery tool 120 to more efficiently detect the mechanical deformation of the surgery tool 120. One or more FBG sensors may be provided in the surgery tool 120. When plural FBG sensors are provided, the FBG sensors may be symmetrically arranged to increase the detection efficiency regarding the mechanical deformation of the surgery tool 120. More specifically, a cylindrical portion may extend from one end of the surgery tool 120, and the plurality of FBG sensors may be symmetrically arranged in close contact with the inner surface or the outer surface of the cylindrical portion.

The temperature sensor 122 is disposed at an inner center of the surgery tool 120 to detect variation of temperature of the surgery tool 120 and accordingly generate a temperature signal. A generally-known temperature sensor or the FBG sensor used for the tactile sensor 121 may be employed as the temperature sensor 122. The FBG sensor may detect temperature using variation of a wavelength of light reflected from some or all of a plurality of fiber optic Bragg gratings, with the variation corresponding to a change of temperature.

The signal converter 130 converts the tactile signal generated by the tactile sensor 121 into an electrical signal

The signal combiner 140 combines the temperature signal with the electrical signal converted from the tactile signal. That is, the signal combiner 140 generates a combination tactile signal containing the temperature signal.

The imager 150 is mounted to a main body of the slave robot 100 and generates a surgery image by imaging the surgery region during the surgery. The slave controller 160 controls operation of the surgery tool 120 corresponding to the control signal received through the slave transceiver 110. In addition, the slave controller 160 may control the slave transceiver 110 to transmit the combination tactile signal combined by the signal combiner 140 and the surgery image generated by the imager 150, to the master robot 200.

The master robot 200 includes a master transceiver 210, the user operator 220, a display 230, and a master controller 240. The master transceiver 210 receives and transmits signals to and from the slave robot 100. The master transceiver 210 receives the tactile signal and the surgery image from the slave robot 100. The display 230 may display the surgery image.

The user operator 220 is mounted to a main body of the master robot 200 and is structured to be pinched or gripped by a hand of a user, for example a surgeon. When the user pinching or gripping the user operator 220 performs a hand closing motion or redirects the user operator 220, the user operator 220 generates a corresponding control signal. Since the control signal is adapted to control operation of the surgery tool 120, the user may operate the user operator 220 by checking the surgery image being displayed through the display 230.

The user operator 220 includes a tactile reproduction actuator 221, a temperature value generator 222 and a signal adjuster 223. The tactile signal received by the master transceiver 210 may contain a temperature signal. Therefore, in this case, the temperature value generator 222 may separate the temperature signal from the tactile signal and generate a temperature value corresponding to the separated temperature signal.

The signal adjuster 223 adjusts a signal level of the tactile signal from which the temperature signal is separated, so that the tactile signal is processable by the master robot 200.

The tactile reproduction actuator 221 reproduces the tactile signal of which the signal level is adjusted by the signal adjuster 223. The tactile reproduction actuator 221 mechanically operates in accordance with the tactile signal; that is, the electrical signal provides tactile feedback regarding the surgery region being sensed by the surgery tool 120. The tactile reproduction actuator 221 may employ any one of a piezoelectric actuator, a pneumatic actuator, and an SMA actuator, however, the tactile reproduction actuator 221 is not limited thereto. Any other actuators or tactile reproduction devices capable of reproducing a tactile signal may also be used as the tactile reproduction actuator 221.

The display 230 may display the tactile signal of which the signal level is adjusted by the signal adjuster 223, and the temperature signal. For example, the temperature signal and the tactile signal may be displayed in the form of numbers or graphs.

The master controller 240 may control the master transceiver 210 to transmit the control signal corresponding to the operation of the user operator 220, to the slave robot 100. Additionally, the master controller 240 controls the tactile reproduction actuator 221 to reproduce the tactile signal, and controls the temperature value generator 222 to generate a temperature value corresponding to the temperature signal.

According to the surgery robot system shown in FIG. 2, a tactile sense generated by contact between the surgery tool 120 and the surgery region may be reproduced by the user operator 220 which contacts the user's hand. Therefore, the user may perform palpation of the surgery region through the tactile feedback even when the surgery robot system is performing the surgery.

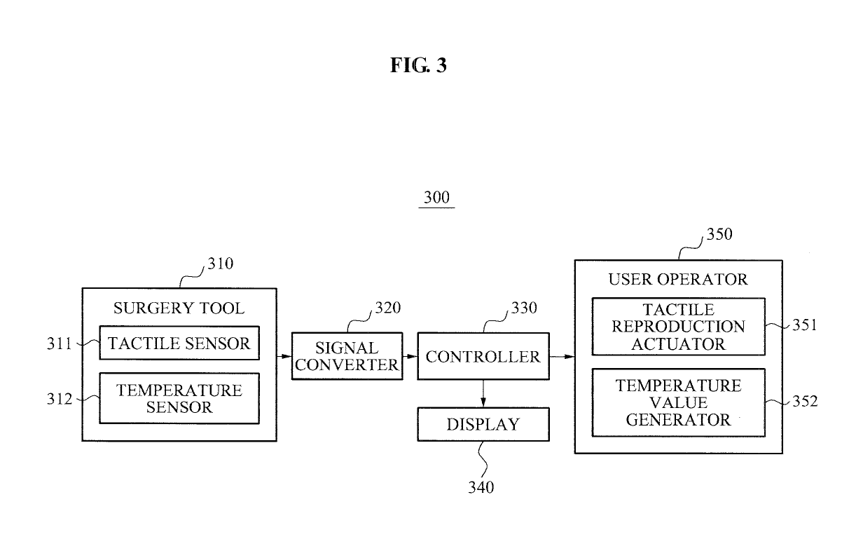

FIG. 3 is a block diagram illustrating a structure of a surgery apparatus 300 according to example embodiments. Referring to FIG. 3, the surgery apparatus 300 refers to an apparatus to perform surgery by operating a surgery tool 310 in accordance with a user operation. The surgery apparatus 300 may be in the form of an apparatus integrally including functions of the slave robot 100 and the master robot 200, like the surgery robot system illustrated with FIGS. 1 and 2. That is, the surgery apparatus 300 may include the surgery tool 310 and a user operator 350 for controlling the surgery tool 310, which are mounted to a main body.

The surgery apparatus 300 may include the surgery tool 310, a signal converter 320, a controller 330, a display 340, and the user operator 350. The user operator 350 may be connected to the surgery tool 300 to generate a control signal for controlling operation of the surgery tool 310 in accordance with a user operation. When the user performs a hand closing motion or redirects the user operator 350 in the state of pinching or gripping the user operator 350, the user operator 350 generates a corresponding control signal.

The controller 330 controls the surgery tool 310 using the control signal generated from the user operator 350. The surgery tool 310 performs an operation corresponding to the control signal, thereby performing the surgery on a patient. The surgery tool 310 includes a tactile sensor 311 and a temperature sensor 312.

While the surgery tool 310 is performing the surgery, the tactile sensor 311 detects contact between the surgery tool 310 and a surgery region of the patient and accordingly generates a tactile signal. At least one tactile sensor 311 may be mounted on an inner surface or an outer surface of the surgery tool 310. The tactile sensor 311 may detect a degree of contact between the surgery tool 310 and the surgery region according to a mechanical deformation of the surgery tool 310, and generate the tactile signal according to the degree of contact being detected.

The temperature sensor 312 may be disposed internally at a center of the surgery tool 310 to detect a variation in temperature of the surgery tool 310 and accordingly generate a temperature signal.

An FBG sensor may be used as the tactile sensor 311 and the temperature sensor 312.

The signal converter 320 converts the tactile signal generated by the tactile sensor 311 into an electrical signal. The signal converter 320 may convert, into an electrical signal, the tactile signal only or a combination tactile signal combined with the temperature signal.

Upon receiving the temperature signal and the tactile signal through the signal converter 320, the controller 330 controls a tactile reproduction actuator 351, a temperature value generator 352, and a display 340 included in the user operator 350. The tactile reproduction actuator 351 reproduces the tactile signal. More specifically, the tactile reproduction actuator 351 may mechanically operate in accordance with the tactile signal which is an electrical signal, thereby providing tactile feedback regarding the surgery region being sensed by the surgery tool 310. The temperature value generator 352 may generate a temperature value corresponding to the temperature signal.

The display 340 may display the tactile signal and the temperature signal. The display 340 may display strength or a region corresponding to the tactile signal in the form of texts or graphs, and display the temperature signal in the form of texts. Also, the display 340 may display a surgery image generated by an imaging device (not shown) during the surgery.

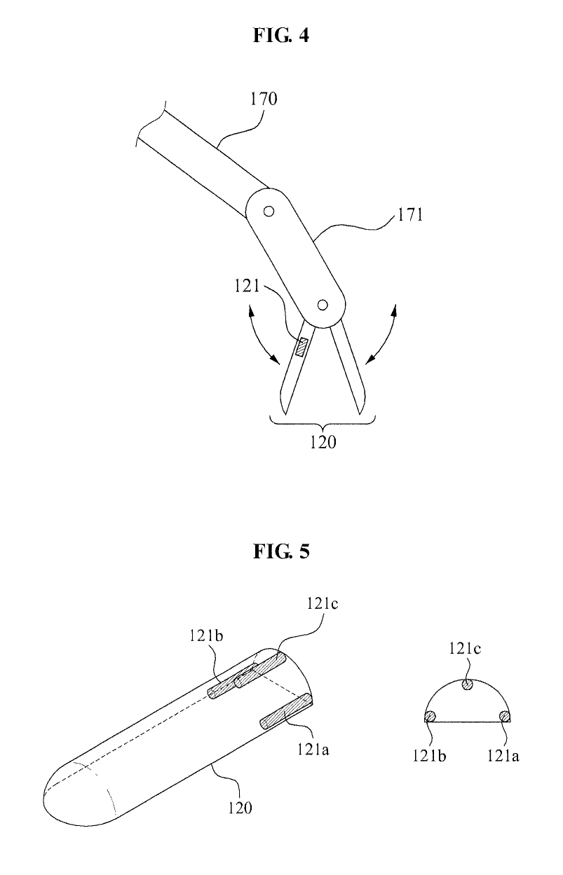

FIG. 4 is a diagram illustrating the surgery tool 120 that generates the tactile signal of the surgery robot system of FIG. 1. Specifically, FIG. 4 is an enlarged view of the surgery tool 120 of FIG. 1. The surgery tool 120 is connected to the robot arm 170 and moved according to a movement of the robot arm 170, thereby being brought into contact with the surgery region. A robot joint 171, which is a part of the robot arm 170, may increase a degree of freedom of the robot arm 170.

Referring to FIG. 4, for example, the surgery tool 120 may be a grasper to grasp specific tissue in the surgery region or a suture. When the user operates the user operator 220 provided to the master robot 220, the surgery tool 120 may perform a pressing motion, a closing motion, or a widening motion in accordance with the user operation, thereby pressing or pulling the surgery region.

The surgery tool 120 includes the tactile sensor 121 mounted therein to detect the contact between the surgery tool 120 and the surgery region and accordingly generate the tactile signal. When the surgery tool 120 contacts the surgery region or pulls the suture, the surgery tool 120 is mechanically deformed by a pressure, tension, attraction, and the like. When the surgery tool 120 is mechanically deformed, the tactile sensor 121 mounted in the surgery tool 120 detects the contact between the surgery tool 120 and the surgery region and measures a degree of the mechanical deformation of the surgery tool 120. Accordingly, the tactile sensor 121 generates the tactile signal. The tactile sensor 121 may be an FBG sensor that generates the tactile signal by detecting strength or temperature using variation of a wavelength of light reflected from a plurality of fiber optic Bragg gratings.

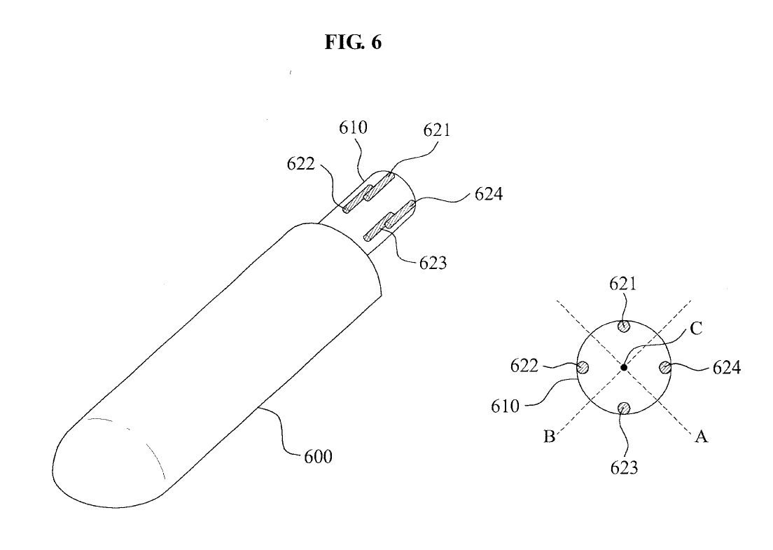

FIGS. 5 to 7 are diagrams illustrating various types of tactile sensors which can be provided to the surgery tool.

FIG. 5 includes a perspective view and a sectional view of the surgery tool 120 which includes a first tactile sensor 121a, a second tactile sensor 121b, and a third tactile sensor 121c. The first tactile sensor 121a and the second tactile sensor 121b may be disposed at both ends of the surgery tool 120 having a semicircular cylinder shape. The third tactile sensor 121c may be disposed in a middle of a curved surface with respect to a width direction. The first tactile sensor 121a, the second tactile sensor 121b and the third tactile sensor 121c may be mounted in close contact with the inner surface of the surgery tool 120 to more efficiently detect the mechanical deformation of the surgery tool 120.

Although FIG. 5 shows the first tactile sensor 121a, the second tactile sensor 121b and the third tactile sensor 121c disposed on the inner surface of the surgery tool 120, the first tactile sensor 121a, the second tactile sensor 121b and the third tactile sensor 121c may be disposed on the outer surface of the surgery tool 120.

FIG. 6 includes a perspective view and a sectional view of a surgery tool 600 including a first tactile sensor 621, a second tactile sensor 622, a third tactile sensor 623, and a fourth tactile sensor 624.

The first through fourth tactile sensors 621-624 may be symmetrically arranged to more efficiently detect the mechanical deformation of the surgery tool 600. To more efficiently detect the mechanical deformation of the surgery tool 600, the surgery tool 600 may include a cylindrical portion 610 extending from one end thereof. For example, the cylindrical portion 610 may extend from one end of the surgery tool 600 where the robot joint 171 is connected as shown in FIG. 4.

The first through fourth tactile sensors 621 to 624 are disposed on an inner surface of the cylindrical portion 610. The first tactile sensor 621 and the third tactile sensor 623 may be symmetrically arranged with respect to a center point C of a cross section of the cylindrical portion 610. Also, the second tactile sensor 622 and the fourth tactile sensor 624 may be symmetrically arranged with respect to the center point C. In addition, the first tactile sensor 621 and the second tactile sensor 622 may be symmetrically arranged and the third tactile sensor 623 and the fourth tactile sensor 624 may be symmetrically arranged with respect to a first straight line A which passes the center point C.

In addition, the first tactile sensor 621 and the fourth tactile sensor 624 may be symmetrically arranged and the second tactile sensor 622 and the third tactile sensor 623 may be symmetrically arranged with respect to a second straight line B which passes the center point C. When the first through fourth tactile sensors 621 to 624 are thus symmetrically arranged, the mechanical deformation of the surgery tool 600 may be more accurately detected.

FIG. 7 includes sectional view of a surgery tool that includes a temperature sensor 715. The surgery tool is structured in the same manner as in FIG. 6. That is, the surgery tool 700 may include a first tactile sensor 711, a second tactile sensor 712, a third tactile sensor 713, and a fourth tactile sensors 714 mounted on an inner surface of a cylindrical portion 700 extending from one end of the surgery tool. The surgery tool may further include a temperature sensor 715.

The first through fourth tactile sensors 711 to 714 may be arranged in the same manner as the tactile sensors shown in FIG. 6. The temperature sensor 715 may be disposed in an inner center of the surgery tool. The FBG sensor used as a tactile sensor may also be used as the temperature sensor 715. Here, the internal center of the surgery tool may be least affected by the mechanical deformation of the surgery tool. When the temperature sensor 715 is mounted in close contact with the inner surface or the outer surface of the surgery tool, the temperature sensor may react more sensitively to the mechanical deformation than to the temperature variation and thus, may fail to accurately detect the temperature. Therefore, the temperature sensor 715 is disposed in the inner center of the surgery tool to detect the temperature variation of the surgery tool and generate a temperature signal.

The slave robot may combine the tactile signal generated by the first through fourth tactile sensors 711 to714 with the temperature signal generated by the temperature sensor 715. Additionally, the slave robot may convert the combination signal into an electrical signal and transmit the electrical signal to the master robot.

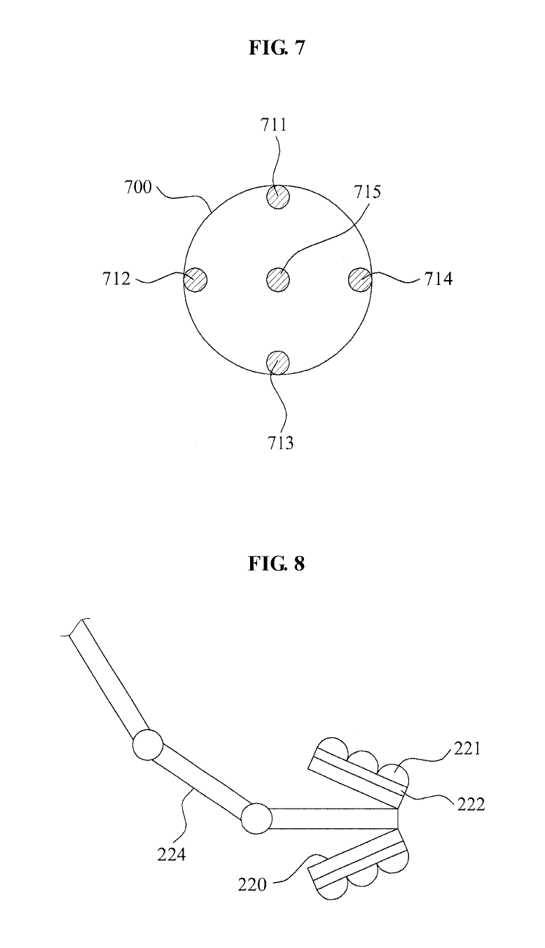

FIG. 8 is a diagram illustrating the user operator 220 that provides tactile feedback. Referring to FIG. 8, the user operator 220 is connected to the master robot by a connector 224. The connector 224 may be moved according to the operation of the user operator 220 by the user. The user operator 220 may be structured for the user to pinch or grip.

When the user pinching or gripping the user operator 220 performs a hand closing motion or redirects the user operator 220, the user operator 220 generates a corresponding control signal. The control signal is adapted to control operation of the surgery tool. The user operator 220 includes the tactile reproduction actuator 221 and the temperature value generator 222.

The tactile reproduction actuator 221 reproduces the tactile signal received from the slave robot. Specifically, the tactile reproduction actuator 221 may be formed on the overall surface which contacts a hand of the user. That is, the tactile reproduction actuator 221 may be formed on the surface to be brought into contact with at least one of a palm, a thumb, an index finger, a middle finger, a ring finger, and a little finger of the user and may reproduce the tactile signal partially or overall. For example, when the tactile signal corresponds to an operation of pinching the surgery region by the surgery tool 120, the tactile reproduction actuator 221 may operate only at parts corresponding to the thumb and the index finger and generate the tactile signal.

The tactile reproduction actuator 221 may generate the tactile signal in consideration of a gripping force applied to the user operator 220 by the user. For example, assuming that the tactile signal is a pressure signal corresponding to an operation of pressing the surgery region and a size of the pressure signal is 1, when the gripping force of the user holding the user operator 220 is 0.5, the tactile reproduction actuator 221 may reproduce a tactile signal corresponding to the pressure of 0.5. However, assuming that the size of the pressure signal is 1, when the gripping force of the user holding the user operator 220 is 0, the tactile reproduction actuator 221 may reproduce a tactile signal corresponding to the pressure of 1.

The temperature value generator 222 separates the temperature signal from the tactile signal and generates a temperature value corresponding to the temperature signal. The temperature value generator 222 may be disposed at a lower portion of the tactile reproduction actuator 221, on the overall surface contacting the hand of the user. Therefore, even when the hand of the user is contacting only a part of the user operator 220, the user is able to feel the temperature generated from the temperature value generator 222.

While performing surgery using the user operator 220, the user may simultaneously feel the tactile sense and the temperature felt by the surgery tool 120 from the surgery region. Therefore, the user may perform palpation through the tactile feedback even when using the surgery robot system. Thus, the user may be aware of a contacting force of surgery tools applied to the surgery region. Consequently, the user may control a force for operating the user operator 220 so that tissues of the surgery region are not damaged. In addition, the user may be able to detect abnormal tissues through the palpation.

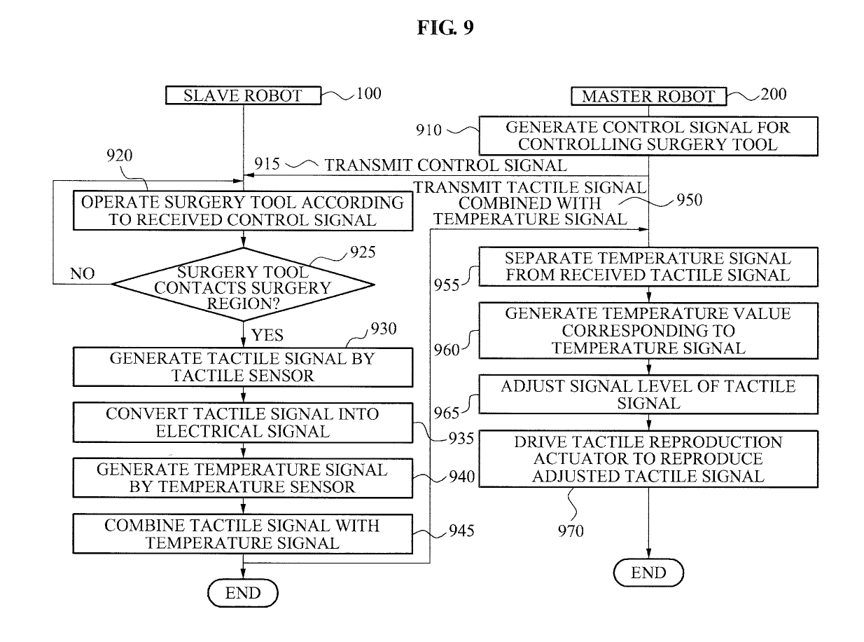

FIG. 9 is a flowchart illustrating a method for providing tactile feedback according to example embodiments. The surgery robot system includes the slave robot 100 mounted with the surgery tool 120, and the master robot 200 to control the operation of the surgery tool 120. The surgery robot system enables the performance of surgery using the robots 100 and 200.

The slave robot 100 and the master robot 200 perform surgery on the patient by transceiving signals with each other, and by providing the tactile feedback.

First, when the user operator 220 is operated, the master robot 200 generates a control signal to control the operation of the surgery tool 120 in operation 910. The master robot 200 transmits the control signal to the slave robot 100 in operation 915. The slave robot 100 operates the surgery tool 120 in accordance with the control signal received from the master robot 200 in operation 920.

Upon detecting contact between the surgery tool 120 and the surgery region in operation 925, the slave robot 100 generates a tactile signal using the tactile sensor 121 in operation 930. The slave robot 100 converts the tactile signal into an electrical signal to be transmitted to the master robot 200 in operation 935. Additionally, the slave robot 100 generates a temperature signal using the temperature sensor 122 in operation 940. In operation 945, the slave robot 100 combines the temperature signal with the tactile signal generated in operation 930. Next, the slave robot 100 transmits the combination tactile signal combined with the temperature signal to the master robot 200 in operation 950.

The master robot 200 separates the temperature signal from the tactile signal received from the slave robot 100 in operation 955. Next, the master robot 200 generates the temperature value corresponding to the separated temperature signal using the temperature value generator 222 provided in the user operator 220 in operation 960. In addition, the master robot 200 adjusts the signal level of the tactile signal not containing the temperature signal in operation 965, such that the tactile signal is processable by the master robot 200, and then drives the tactile reproduction actuator 221 to reproduce the level-adjusted tactile signal in operation 970.

The master robot 200 may adjust the signal level of the tactile signal using a predetermined algorithm. For example, for adjustment of the signal level, the tactile signal may be mapped to a linear function or a log function.

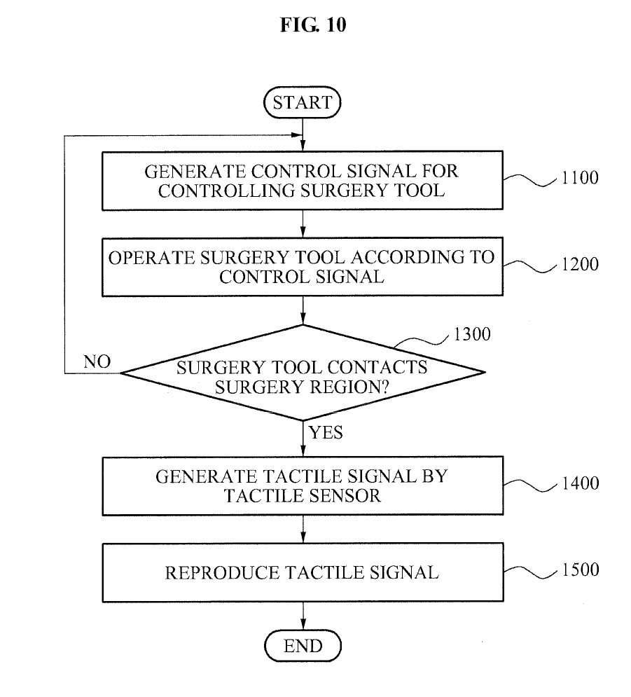

FIG. 10 is a flowchart illustrating a method for providing a tactile feedback according to other example embodiments. The method of FIG. 10 may be embodied by the surgery apparatus 300 shown in FIG. 3. Referring to FIG. 10, when a user operator 350 connected to a main body of the surgery apparatus 300 is operated, the surgery apparatus 300 generates a control signal for controlling operation of a surgery tool 310, in operation 1100.

Next, the surgery apparatus 300 operates the surgery tool 310 in accordance with the control signal in operation 1200. When the user operator 350 is moved rightward by a first distance by the user operation, the surgery apparatus 300 may generate a control signal denoting the rightward moved distance. In addition, according to the control signal, the surgery apparatus 300 operates the surgery tool 310 rightward by the first distance.

When the surgery tool 310 contacts the surgery region during the surgery in operation 1300, the surgery apparatus 300 generates a tactile signal using the tactile sensor 311 provided in the surgery tool 310 in operation 1400. The surgery apparatus 300 generates the tactile signal using the tactile reproduction actuator 351 of the user operator 350 in operation 1500. The surgery apparatus 300 generates the tactile signal by detecting the contact between the surgery tool and the surgery region, and provides the user with tactile feedback by reproducing the tactile signal. By using tactile feedback, the user may control the force applied to the tissues of the surgery region and detect abnormal tissues, invisible to the naked eye, by palpation.

The methods according to the above-described example embodiments may be recorded in non-transitory computer-readable media including program instructions to implement various operations embodied by a computer. The media may also include, alone or in combination with the program instructions, data files, data structures, and the like. The program instructions recorded on the media may be those specially designed and constructed for the purposes of the example embodiments, or they may be of any kind of well-known program instructions available to those having skill in the computer software arts. Examples of non-transitory computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The media may be transfer media such as optical lines, metal lines, or waveguides including a carrier wave for transmitting a signal designating the program command and the data construction. Examples of program instructions include both machine code, such as produced by a compiler, and files containing higher level code that may be executed by the computer using an interpreter. The described hardware devices may be configured to act as one or more software modules in order to perform the operations of the above-described example embodiments, or vice versa.

Although example embodiments have been shown and described, it will be appreciated by those skilled in the art that changes may be made in these example embodiments without departing from the principles and spirit of the disclosure, the scope of which is defined in the claims and their equivalents.

What is claimed is:

1. A method of providing tactile feedback, comprising:
generating a control signal to control operation of a surgery tool;
operating the surgery tool in accordance with the control signal;
generating a contact signal using at least one sensor in the surgery tool when the surgery tool contacts a surgery region; and
reproducing the contact signal.

2. The method of claim 1, wherein the generating the contact signal comprises converting the conatct signal generated from the at least one sensor into an electrical signal.

3. The method of claim 1, wherein the generating the contactsignal comprises detecting a variation of temperature of the surgery tool by a temperature sensor and generating a temperature signal.

4. The method of claim 1, wherein the generating the contact signal comprises detecting a degree of contact between the surgery tool and the surgery region according to a mechanical deformation of the surgery tool, and generating a tactile signal as the contact signal according to the degree of contact being detected.

5. The method of claim 3, wherein the reproducing the contact signal comprises:
generating a temperature value corresponding to the temperature signal by driving a temperature value generator; and
displaying the contact signal and the temperature signal.
